(19) United States
(12) Patent Application Publication
(10) Pub. No.: US 2023/0357280 A1
(43) Pub. Date: Nov. 9, 2023

(54) REACTIVE GRANULATION OF CALCIUM CITRATE MALATE, DICALCIUM MALATE, AND DIMAGNESIUM MALATE

(71) Applicant: Balchem Corporation, New Hampton, NY (US)

(72) Inventors: Ren A. Gonzalez, Nibley, UT (US); Tod Jenness, New Hampton, NY (US)

(21) Appl. No.: 18/195,239

(22) Filed: May 9, 2023

Related U.S. Application Data
(60) Provisional application No. 63/339,732, filed on May 9, 2022.

(51) Int. Cl. C07F 3/04; A61K 9/20

(52) U.S. Cl. CPC C07F 3/04; A61K 9/2077

(57) ABSTRACT

The present invention provides compositions comprising granules of calcium citrate malate, compositions comprising granules of dicalcium malate, compositions comprising granules of dimagnesium malate, methods for preparing these compositions, and dosage formulations of these compositions.

### CROSS-REFERENCE TO RELATED APPLICATIONS

This application claims priority to U.S. Provisional Application No. 63/339,732 entitled “REACTIVE GRANULATION OF CALCIUM CITRATE MALATE, DICALCIUM MALATE, AND DIMAGNESIUM MALATE”, filed on May 9, 2022, the entire contents of which are incorporated by reference herein.

### FIELD OF THE INVENTION

The present disclosure generally relates to compositions comprising granules of calcium citrate malate, comprising granules of dicalcium malate, comprising granules of or dimagnesium malate; methods of preparing these compositions, and dosage formulations (dosage forms) comprising these compositions. These compositions comprising granules of calcium citrate malate, dicalcium malate, or dimagnesium malate have a high water content and may be easily converted into various dosage formulations.

### BACKGROUND OF THE INVENTION

Magnesium is a mineral that is needed in humans and other warm-blooded animals for bone, protein, and fatty acid formation. Magnesium is also involved in the formation of new cells, activating certain vitamins, relaxing muscles, clotting blood, and forming ATP. Benefits of magnesium include improvement of memory and reflexes, reducing of fatigue, and promoting proper development of thyroid hormones, skeletal, reproductive, and central nervous systems. People with diabetes often have magnesium levels that are lower than normal compared with those who have normal glucose tolerance. Supplementation of magnesium can help maintain health in some of these areas, as well as help in overcoming some of these problems. Typically, many people do not consume enough magnesium in their diets. Calcium, on the other hand, is the most abundant mineral in the human body. Of the calcium contained in the average body, about 99% is located in the bones, including the teeth. Calcium is needed to form bones and teeth and is also required for blood clotting, transmission of signals in nerve cells, and muscle contraction. Calcium supplementation is believed to reduce the incidence of osteoporosis.

Choosing a form of magnesium and/or calcium for supplementation has been a source of some confusion in the industry. Calcium carbonate is one form of calcium that is widely used but is not believed to be absorbed as well as some other forms. Calcium citrate provides a form that is believed to be better absorbed than calcium carbonate. Calcium citrate malate (CCM) is believed to be absorbed more fully than calcium carbonate as well.

Malic acid is an organic dicarboxylic acid that is naturally occurring. Malic acid plays a role in the complex process of deriving ATP (the energy currency that runs the body) from food. Malic acid is found in a wide variety of fruits (including richly in apples) and vegetables. As malic acid is already found abundantly in humans and other warm-blooded animals, it can be administered without adverse effects. Further, there is some evidence that malic acid supplementation can be helpful to human nutrition.

A high water content and high water activity in various mineral compositions can degrade the effectiveness, handleability, and processability of these mineral compositions. The high water content and high water activity can provide a medium for various pathogens to grow, allow undesirable interactions with other components, and reduce the shelf life of the mineral supplement.

In contrast, a low water content and low water activity in mineral compositions may increase the shelf life of these mineral supplements and reduce component interactions, but the low water content and low water activity can also increase the number of manufacturing steps needed to obtain these low water compositions in various dosage forms and may reduce the handleability and processability of these supplements. These manufacturing steps may be additional drying steps, utilization of spray drying, additional manufacturing equipment to capture or screen powders, and additional manufacturing equipment and steps, such as roller compaction, to convert these low water and low water activity materials into suitable dosage forms. In some cases, direct processing of low water content and low water activity mineral compositions into granulated forms to further process and convert into mineral supplement dosage forms cannot be directly achieved. To increase handleability and processability of these low water content and low water activity mineral compositions, and convert them to desirable granulated forms, various excipients such as lubricants and compression aids may be needed, which reduces the potency of the original mineral compositions.

Appropriate water content and water activity in these mineral supplements could provide the mineral supplements with a long shelf-life, extended effectiveness, inhibit the growth of pathogens, and provide low-cost methods to convert these mineral supplements into various dosage formulations.

### SUMMARY OF THE INVENTION

Provided herein are compositions comprising granules of calcium citrate malate. The calcium citrate malate has a mol ratio of calcium:malate:citrate of about 6:3:2 and a water content of at least 5.0 wt %. In some embodiments, the composition further comprises at least one excipient. In some embodiments, the granules have a calcium weight percentage of at least 19 wt %. In some embodiments, the granules of calcium citrate malate have a particle size ranging from about 50 microns to about 1500 microns. In some embodiments, the granules have a bulk density of at least 0.70 g/mL. In some embodiments, the composition comprises no more than 5.0 wt % silica. In some embodiments, the composition does not contain silica.

Further provided herein are compositions comprising granules of dicalcium malate. The dicalcium malate has a mol ratio of about 2:1 and a water content of at least 5.0 wt %. In some embodiments, the composition further comprises at least one excipient. In some embodiments, the granules have a calcium weight percentage of at least 27 wt %. In some embodiments, the granules of calcium citrate malate have a particle size ranging from about 50 microns to about 1500 microns. In some embodiments, the granules have a bulk density of at least 0.70 g/mL. In some embodiments, the composition comprises no more than 5.0 wt % silica. In some embodiments, the composition does not contain silica.

Further provided herein are compositions comprising granules of dimagnesium malate. The dimagnesium malate has a mol ratio of magnesium to malate of about 2:1 and a water content of at least 2.0 wt %. In some embodiments, the composition further comprises at least one excipient. In some embodiments, the granules have a magnesium weight percentage of at least 18 wt %. In some embodiments, the granules of calcium citrate malate have a particle size ranging from about 50 microns to about 1500 microns. In some embodiments, the granules have a bulk density of at least 0.70 g/mL. In some embodiments, the composition comprises no more than 5.0 wt % silica. In some embodiments, the composition does not contain silica.

Further provided herein are methods for preparing granules of calcium citrate malate. The methods generally comprise: (a) contacting citric acid and malic acid forming a particulate mixture; (b) adding water to the particulate mixture from step (a) forming a slurry; (c) contacting a calcium base with the slurry from step (b); (d) reacting to form the granules of calcium citrate malate; and (e) isolating the granules of calcium citrate malate. The granules of calcium citrate malate have a water content of at least 5.0 wt %.

Further provided herein are methods for preparing granules dicalcium malate. The methods generally comprise: (a) adding water to a calcium base forming a slurry; (b) contacting the slurry from step (a) with malic acid causing a reaction between the malic acid and the calcium base forming a second slurry; (c) repeating step (b) until the calcium base and malic acid fully react forming granules of dicalcium malate; and (d) isolating the granules of dicalcium malate. The granules of dicalcium malate have a water content of at least 5.0 wt %.

Further provided herein are methods for preparing granules of dimagnesium malate. The methods generally comprise: (a) contacting a magnesium base and a malic acid forming a particulate mixture; (b) adding water to the particulate mixture from step (a) causing a partial reaction between the malic acid and the magnesium base forming a slurry; (c) repeating step (b) until the malic acid and the magnesium base fully react forming granules; and (d) isolating the granules of dimagnesium malate. The granules of dimagnesium malate have a water content of at least 2.0 wt %.

Further provided herein are dosage formulations comprising granules of calcium citrate malate. In some embodiments, the dosage formulation is a tablet comprising compressed granules of calcium citrate malate.

Further provided herein are dosage formulations comprising granules of dicalcium malate. In some embodiments, the dosage formulation is a tablet comprising compressed granules of dicalcium malate.

Further provided herein are dosage formulations comprising granules of dimagnesium malate. In some embodiments, the dosage formulation is a tablet comprising compressed granules of dimagnesium malate.

### BRIEF DESCRIPTION OF THE FIGURES

FIG. 1 is a photograph comparing a spray-dried powder of calcium citrate malate (left) versus granules from a reactive granulation of calcium citrate malate (right).

FIG. 2 is a photograph comparing a spray-dried powder of dicalcium malate (left) versus granules from a reactive granulation of dicalcium malate (right).

FIG. 3 is a photograph comparing a spray-dried powder of dimagnesium malate (left) versus granules from a reactive granulation of dimagnesium malate (right).

### DETAILED DESCRIPTION OF THE INVENTION

Provided herein are compositions and methods for preparing granules of calcium citrate malate, granules of dicalcium malate, and granules of dimagnesium malate from a reactive granulation process. Advantageously, these compositions can be efficiently prepared and easily converted into various dosage formulations. The methods disclosed herein include improvements which allow the compositions to be easily prepared, highly reproducible, and a low manufacturing cost.

### (I) Compositions Comprising Granules of Calcium Citrate Malate

The present disclosure encompasses compositions comprising granules of calcium citrate malate having a mole ratio of calcium:malate:citrate of about 6:3:2. The granules of calcium citrate malate may have a high water content. As used in this section, a composition comprising granules of dicalcium malate with a high water content may have a water content of at least 5 wt % or greater. These properties of the granules allow the calcium citrate malate compositions to be easily converted into various dosage formulations.

Generally, the compositions comprising granules of calcium citrate malate have a water content of at least 5.0 wt % or greater. In various embodiments, the granules of calcium citrate malate have a water content of at least 5.0 wt % or greater, from about 5.0 wt % to about 5.5 wt %, from about 5.5 wt % to about 6.0 wt %, from about 6.0 wt % to about 6.5 wt %, from about 6.5 wt % to about 7.0 wt %, from about 7.0 wt % to about 7.5 wt %, from about 7.5 wt % to about 8.0 wt %, from about 8.0 wt % to about 8.5 wt %, from about 8.5 wt % to about 9.0 wt %, from about 9.0 wt % to about 9.5 wt %, from about 9.5 wt % to about 10.0 wt %, or greater than about 10.0 wt % including all subranges in-between. In one embodiment, the granules of calcium citrate malate have a water content from at least 5.0 wt % to about 10.0 wt %. In yet another non-limiting embodiment, the granules of calcium citrate malate have a water content of up to about 30.0 wt %.

In one or more embodiments, the water content in the granules of calcium citrate malate may range from 5 wt % to 6 wt %, 5 wt % to 7 wt %, 5 wt % to 8 wt %, 5 wt % to 9 wt %, 5 wt % to 10 wt %, 6 wt % to 10 wt %, 7 wt % to 10 wt %, 8 wt % to 10 wt %, or 9 wt % to 10 wt %. In yet other embodiments, the granules of calcium citrate malate may range from 6 wt % to 9 wt %, 7 wt % to 9 wt %, 8 wt % to 9 wt %, or 7 wt % to 8 wt %.

In general, the compositions comprising granules of calcium citrate malate have a calcium content (calcium percentage, Ca %) of at least 19 wt % or greater. In various embodiments, the granules of calcium citrate have a calcium content of at least 19 wt %, at least 20 wt %, at least 21 wt %, at least 22 wt %, at least 23 wt %, at least 24 wt %, at least 25 wt %, at least 26 wt %, at least 27 wt %, at least 28 wt %, at least 29 wt %, at least 30 wt %, at least 31 wt %, at least 32 wt %, at least 33 wt %, at least 34 wt %, at least 35 wt %, at least 36 wt %, at least 37 wt %, at least 38 wt %, at least 39 wt %, at least 40 wt %, at least 41 wt %, at least 42 wt %, at least 43 wt %, at least 44 wt %, at least 45 wt %, at least 46 wt %, at least 47 wt %, at least 48 wt %, at least 49 wt %, or about 50 wt %. In an embodiment, the compositions comprising granules of calcium citrate malate have a calcium content (Ca %) of at least 19 wt % to about 30 wt %.

The compositions comprising granules of calcium citrate malate have a particle size ranging from about 50 microns to about 1500 microns. In various embodiments, the compositions comprising granules of calcium citrate malate have a particle size ranging from about 50 microns to about 1500 microns, from about 150 microns to about 1200 microns, from about 500 microns to about 1000 microns, or from about 700 microns to about 850 microns, including all subranges in-between. In one embodiment, at least 75 wt % of the granules of calcium citrate malate have a particle size ranging from about 90 microns to about 1000 microns including all subranges in-between.

In one or more embodiments, the granules of calcium citrate malate may have a particle size ranging from 50 microns to 100 microns, from 50 microns to 200, from 50 microns to 300 microns, from 50 microns to 400 microns, from 50 microns to 500 microns, from 50 microns to 600, from 50 microns to 700 microns, from 50 microns to 800 microns, from 50 microns to 900 microns, from 50 microns to 1000, from 50 microns to 1100 microns, from 50 microns to 1200 microns, from 50 microns to 1300 microns, from 50 microns to 1400 microns, from 50 microns to 1500 microns, from 100 microns to 1500, from 200 microns to 1500 microns, from 300 microns to 1500 microns, from 400 microns to 1500 microns, from 500 microns to 1500, from 600 microns to 1500 microns, from 700 microns to 1500 microns, from 800 microns to 1500 microns, from 900 microns to 1500, from 1000 microns to 1500 microns, from 1100 microns to 1500 microns, from 1200 microns to 1500 microns, from 1300 microns to 1500 microns, or from 1400 microns to 1500 microns. In yet other embodiments, the granules of calcium citrate malate may have a particle size ranging from 500 microns to 1000 microns, 600 microns to 900 microns, or 700 microns to 800 microns.

In a preferred embodiment, 85 wt % to 95 wt % of the granules of calcium citrate malate may have a particle size ranging from about 90 microns to about 1000 microns.

Generally, the compositions comprising granules of calcium citrate malate have a bulk density of at least 0.70 g/mL or greater. In various embodiments, the granules of calcium citrate malate have a bulk density of at least 0.70 g/mL, at least 0.71 g/mL, at least 0.72 g/mL, at least 0.73 g/mL, at least 0.74 g/mL, at least 0.75 g/mL, at least 0.76 g/mL, at least 0.77 g/mL, at least 0.78 g/mL, at least 0.79 g/mL, at least 0.80 g/mL, at least 0.81 g/mL, at least 0.82 g/mL, at least 0.83 g/mL, at least 0.84 g/mL, at least 0.85 g/mL, at least 0.86 g/mL, at least 0.87 g/mL, at least 0.88 g/mL, at least 0.89 g/mL, at least 0.90 g/mL, at least 0.91 g/mL, at least 0.92 g/mL, at least 0.93 g/mL, at least 0.94 g/mL, at least 0.95 g/mL, at least 0.96 g/mL, at least 0.97 g/mL, at least 0.98 g/mL, at least 0.99 g/mL, at least 1.00 g/mL, at least 1.01 g/mL, at least 1.02 g/mL, at least 1.03 g/mL, at least 1.04 g/mL, at least 1.05 g/mL, at least 1.06 g/mL, at least 1.07 g/mL, at least 1.08 g/mL, at least 1.09 g/mL, or at least 1.10 g/mL or greater.

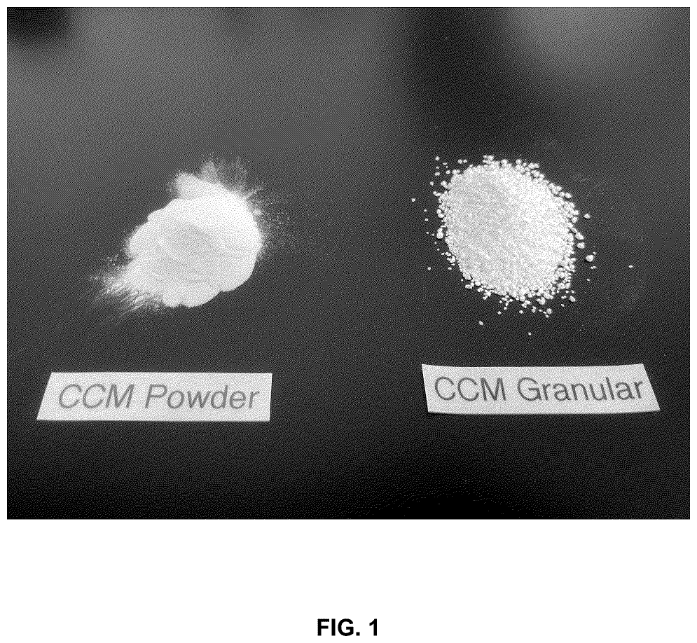

FIG. 1 shows a photographic comparison of the granules of calcium citrate malate (right) from the reactive granulation as compared to powders prepared through other conventional methods (left).

The composition comprising granules of calcium citrate malate may further comprise a pharmaceutically acceptable excipient. Non-limiting examples of suitable pharmaceutically acceptable excipients include a diluent, a binder, a filler, a buffering agent, a pH modifying agent, a disintegrant, a dispersant, a preservative, a lubricant, taste-masking agent, a flavoring agent, a coloring agent, or a combination thereof. The amount and types of excipients utilized to form pharmaceutical compositions may be selected according to known principles of pharmaceutical science.

In one embodiment, the excipient may be a diluent. The diluent may be compressible (i.e., plastically deformable) or abrasively brittle. Non-limiting examples of suitable compressible diluents include microcrystalline cellulose (MCC), cellulose derivatives, cellulose powder, cellulose esters (i.e., acetate and butyrate mixed esters), ethyl cellulose, methyl cellulose, hydroxypropyl cellulose, hydroxypropyl methylcellulose, sodium carboxymethylcellulose, corn starch, phosphorated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, maltitol, sorbitol, xylitol, maltodextrin, or trehalose. Non-limiting examples of suitable abrasively brittle diluents include dibasic calcium phosphate (anhydrous or dihydrate), calcium phosphate tribasic, calcium carbonate, or magnesium carbonate.

In another embodiment, the excipient may be a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, polypeptides, oligopeptides, or combinations thereof.

In another embodiment, the excipient may be a filler. Suitable fillers include, but are not limited to, carbohydrates, inorganic compounds, polyvinylpyrrolidone, and combinations thereof. Representative examples of fillers include, but are not limited to, calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, and sorbitol, and combinations thereof.

In still another embodiment, the excipient may be a buffering agent. Representative examples of suitable buffering agents include, but are not limited to, phosphates, carbonates, citrates, tris buffers, and buffered saline salts (e.g., Tris buffered saline, or phosphate buffered saline).

In various embodiments, the excipient may be a pH modifier. Representative examples of pH modifying agents include, but are not limited to, sodium carbonate, sodium bicarbonate, sodium citrate, citric acid, and phosphoric acid, and combinations thereof.

In a further embodiment, the excipient may be a disintegrant. The disintegrant may be non-effervescent or effervescent. Representative examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pectin, and tragacanth, and combinations thereof. Non-limiting examples of suitable effervescent disintegrants include sodium bicarbonate in combination with citric acid and sodium bicarbonate in combination with tartaric acid.

In yet another embodiment, the excipient may be a dispersant or dispersing enhancing agent. Representative examples of dispersants may include, but are not limited to, starch, alginic acid, polyvinylpyrrolidones, guar gum, kaolin, bentonite, purified wood cellulose, sodium starch glycolate, amorphous silicate, and microcrystalline cellulose, and combinations thereof.

In another alternate embodiment, the excipient may be a preservative. Representative examples of suitable preservatives include, but are not limited to, antioxidants, such as butylated hydroxyanisole (BHA), butylated hydroxytoluene (BHT), vitamin A, vitamin C, vitamin E, retinyl palmitate, citric acid, and sodium citrate; chelators such as ethylenediaminetetraacetic acid (EDTA) and ethylene glycol tetraacetic acid (EGTA); and antimicrobials, such as parabens, chlorobutanol, and phenol, and combinations thereof.

In a further embodiment, the excipient may be a lubricant. Representative examples of suitable lubricants include, but are not limited to, minerals such as talc, silica, and sodium lauryl sulfate; and fats such as vegetable stearin, magnesium stearate, and stearic acid, and combinations thereof.

In yet another embodiment, the excipient may be a taste-masking agent. Taste-masking materials include cellulose ethers; polyethylene glycols; polyvinyl alcohol; polyvinyl alcohol and polyethylene glycol copolymers; monoglycerides or triglycerides; acrylic polymers; mixtures of acrylic polymers with cellulose ethers; cellulose acetate phthalate; and combinations thereof.

In an alternate embodiment, the excipient may be a flavoring agent. Representative examples of suitable flavoring agents include, but are not limited to, synthetic flavor oils and flavoring aromatics and/or natural oils, extracts from plants, leaves, flowers, fruits, and combinations thereof.

In still a further embodiment, the excipient may be a coloring agent. Representative examples of color additives include, but are not limited to, food, drug, and cosmetic colors (FD&C), drug and cosmetic colors (D&C), and external drug and cosmetic colors (Ext. D&C).

The weight fraction of the excipient or combination of excipients in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2% or less, or about 1% or less of the total weight of the composition.

In some embodiments, the composition may comprise no more than 5.00 wt % silica. Without wishing to be bound by theory, the presence of silica may prevent the increase of the water content in the composition. In some non-limiting embodiments, the composition may comprise no more than 5.0 wt % silica, no more than 4.5 wt % silica, no more than 4.0 wt % silica, no more than 3.5 wt % silica, no more than 3.0 wt % silica, no more than 2.5 wt % silica, no more than 2.0 wt % silica, no more than 1.5 wt % silica, no more than 1.0 wt % silica, no more than 0.5 wt % silica, no more than 0.25 wt % silica, no more than 0.1 wt % silica, no more than 0.09 wt % silica, no more than 0.08 wt % silica, no more than 0.07 wt % silica, no more than 0.06 wt % silica, no more than 0.05 wt % silica, no more than 0.04 wt % silica, no more than 0.03 wt % silica, no more than 0.02 wt % silica, no more than 0.01 wt % silica, or no more than 0.001 wt % silica. In some additional non-limiting embodiments, the composition may be free of silica.

### (II) Compositions Comprising Granules of Dicalcium Malate

The present disclosure also encompasses compositions comprising granules of dicalcium malate having a mole ratio of calcium:malate of about 2:1. The granules of dicalcium malate may have a high water content. As used in this section, a composition comprising granules of dicalcium malate with a high water content may have a water content of at least 5 wt % or greater. These properties of the granules allow the dicalcium malate to be easily converted into various dosage formulations.

Generally, the compositions comprising granules of dicalcium malate have a water content of at least 5.0 wt % or greater. In various embodiments, the granules of dicalcium malate have a water content of at least 5.0 wt % or greater, from about 5.0 wt % to about 5.5 wt %, from about 5.5 wt % to about 6.0 wt %, from about 6.0 wt % to about 6.5 wt %, from about 6.5 wt % to about 7.0 wt %, from about 7.0 wt % to about 7.5 wt %, from about 7.5 wt % to about 8.0 wt %, from about 8.0 wt % to about 8.5 wt %, from about 8.5 wt % to about 9.0 wt %, from about 9.0 wt % to about 9.5 wt %, from about 9.5 wt % to about 10.0 wt %, or greater than about 10.0 wt % including all subranges in-between. In one embodiment, the granules of dicalcium malate have a water content from at least 5.0 wt % to about 10 wt %. In yet another non-limiting embodiment, the granules of dicalcium malate have a water content of up to about 30.0 wt %.

In one or more embodiments, the water content in the granules of dicalcium malate may range from 5 wt % to 6 wt %, 5 wt % to 7 wt %, 5 wt % to 8 wt %, 5 wt % to 9 wt %, 5 wt % to 10 wt %, 6 wt % to 10 wt %, 7 wt % to 10 wt %, 8 wt % to 10 wt %, or 9 wt % to 10 wt %. In yet other embodiments, the granules of dicalcium malate may range from 6 wt % to 9 wt %, 7 wt % to 9 wt %, 8 wt % to 9 wt %, or 7 wt % to 8 wt %.

In general, the compositions comprising granules of dicalcium malate have a calcium content (calcium percentage, Ca %) of at least 27 wt % or greater. In various embodiments, the granules of dicalcium malate have a calcium content of at least 27 wt %, at least 28 wt %, at least 29 wt %, at least 30 wt %, at least 31 wt %, at least 32 wt %, at least 33 wt %, at least 34 wt %, at least 35 wt %, at least 36 wt %, at least 37 wt %, at least 38 wt %, at least 39 wt %, at least 40 wt %, at least 41 wt %, at least 42 wt %, at least 43 wt %, at least 44 wt %, at least 45 wt %, at least 46 wt %, at least 47 wt %, at least 48 wt %, at least 49 wt %, or about 50 wt %. In an embodiment, the compositions comprising granules of dicalcium malate have a calcium content (Ca %) of at least 27 wt % to about 35 wt %.

The compositions comprising granules of dicalcium malate have a particle size ranging from about 50 microns to about 1500 microns. In various embodiments, the compositions comprising granules of dicalcium malate have a particle size ranging from about 50 microns to about 1500 microns, from about 150 microns to about 1200 microns, from about 500 microns to about 1000 microns, or from about 700 microns to about 850 microns, including all subranges in-between. In one embodiment, at least 75 wt % of the granules of dicalcium malate have a particle size ranging from about 90 microns to about 1000 microns including all subranges in-between.

In one or more embodiments, the granules of dicalcium malate may have a particle size ranging from 50 microns to 100 microns, from 50 microns to 200, from 50 microns to 300 microns, from 50 microns to 400 microns, from 50 microns to 500 microns, from 50 microns to 600, from 50 microns to 700 microns, from 50 microns to 800 microns, from 50 microns to 900 microns, from 50 microns to 1000, from 50 microns to 1100 microns, from 50 microns to 1200 microns, from 50 microns to 1300 microns, from 50 microns to 1400 microns, from 50 microns to 1500 microns, from 100 microns to 1500, from 200 microns to 1500 microns, from 300 microns to 1500 microns, from 400 microns to 1500 microns, from 500 microns to 1500, from 600 microns to 1500 microns, from 700 microns to 1500 microns, from 800 microns to 1500 microns, from 900 microns to 1500, from 1000 microns to 1500 microns, from 1100 microns to 1500 microns, from 1200 microns to 1500 microns, from 1300 microns to 1500 microns, or from 1400 microns to 1500 microns. In yet other embodiments, the granules of dicalcium malate may have a particle size ranging from 500 microns to 1000 microns, 600 microns to 900 microns, or 700 microns to 800 microns.

In a preferred embodiment, 85 wt % to 95 wt % of the granules of dicalcium malate may have a particle size ranging from about 90 microns to about 1000 microns.

Generally, the compositions comprising granules of dicalcium malate have a bulk density greater of at least 0.70 g/mL. In various embodiments the granules of dicalcium malate have a bulk density of at least 0.70 g/mL, at least 0.71 g/mL, at least 0.72 g/mL, at least 0.73 g/mL, at least 0.74 g/mL, at least 0.75 g/mL, at least 0.76 g/mL, at least 0.77 g/mL, at least 0.78 g/mL, at least 0.79 g/mL, at least 0.80 g/mL, at least 0.81 g/mL, at least 0.82 g/mL, at least 0.83 g/mL, at least 0.84 g/mL, at least 0.85 g/mL, at least 0.86 g/mL, at least 0.87 g/mL, at least 0.88 g/mL, at least 0.89 g/mL, at least 0.90 g/mL, at least 0.91 g/mL, at least 0.92 g/mL, at least 0.93 g/mL, at least 0.94 g/mL, at least 0.95 g/mL, at least 0.96 g/mL, at least 0.97 g/mL, at least 0.98 g/mL, at least 0.99 g/mL, at least 1.00 g/mL, at least 1.01 g/mL, at least 1.02 g/mL, at least 1.03 g/mL, at least 1.04 g/mL, at least 1.05 g/mL, at least 1.06 g/mL, at least 1.07 g/mL, at least 1.08 g/mL, at least 1.09 g/mL, or at least 1.10 g/mL or greater.

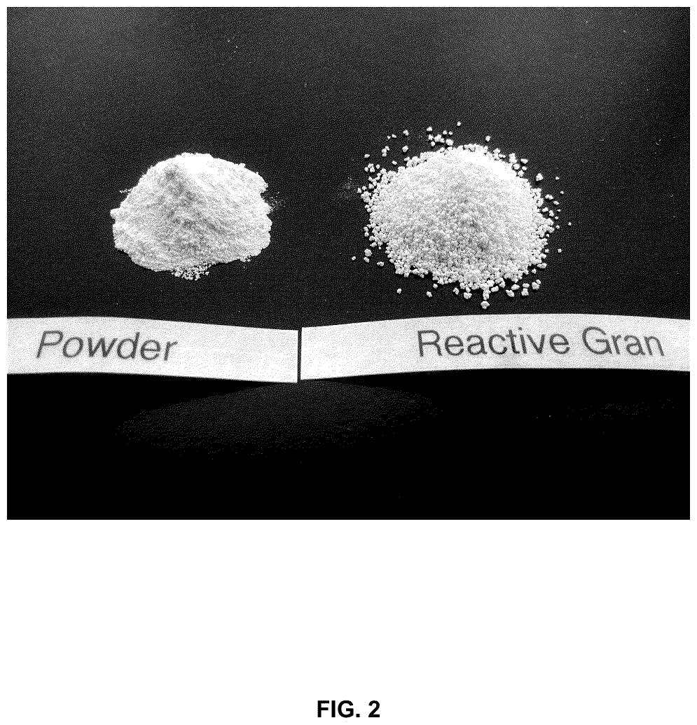

FIG. 2 shows a photographic comparison of the granules of dicalcium malate (right) from the reactive granulation as compared to powders prepared through other conventional methods (left).

The composition comprising granules of dicalcium malate may further comprise at least one pharmaceutically acceptable excipient. Examples of pharmaceutically acceptable excipients are detailed above in Section (1).

In some embodiments, the composition may comprise no more than 5.0 wt % silica. In some non-limiting embodiments, the composition may comprise no more than 5.0 wt % silica, no more than 4.5 wt % silica, no more than 4.0 wt % silica, no more than 3.5 wt % silica, no more than 3.0 wt % silica, no more than 2.5 wt % silica, no more than 2.0 wt % silica, no more than 1.5 wt % silica, no more than 1.0 wt % silica, no more than 0.5 wt % silica, no more than 0.25 wt % silica, no more than 0.1 wt % silica, no more than 0.09 wt % silica, no more than 0.08 wt % silica, no more than 0.07 wt % silica, no more than 0.06 wt % silica, no more than 0.05 wt % silica, no more than 0.04 wt % silica, no more than 0.03 wt % silica, no more than 0.02 wt % silica, no more than 0.01 wt % silica, or no more than 0.001 wt % silica. In some additional non-limiting embodiments, the composition may be free of silica.

### (III) Compositions Comprising Granules of Dimagnesium Malate

The present disclosure additionally encompasses compositions of granules of dimagnesium malate having a mole ratio of magnesium:malate of about 2:1. The granules of dimagnesium malate may have a high water content. As used in this section, a composition comprising granules of dimagnesium malate with a high water content may have a water content of at least 2.0 wt % or greater. These properties of the granules allow the dimagnesium malate to be easily converted into various dosage formulations.

Generally, the compositions comprising granules of dimagnesium malate have a water content of at least 2.0 wt % or greater. In various embodiments, the granules of dimagnesium malate have a water content of at least 2.0 wt % or greater, from about 2.0 wt % to about 2.5 wt %, from about 2.5 wt % to about 3.0 wt %, from about 3.0 wt % to about 3.5 wt %, from about 3.5 wt % to about 4.0 wt %, from about 4.0 wt % to about 4.5 wt %, from about 4.5 wt % to about 5.0 wt %, from about 5.0 wt % to about 5.5 wt %, from about 5.5 wt % to about 6.0 wt %, from about 6.0 wt % to about 6.5 wt %, from about 6.5 wt % to about 7.0 wt %, from about 7.0 wt % to about 7.5 wt %, from about 7.5 wt % to about 8.0 wt %, from about 8.0 wt % to about 8.5 wt %, from about 8.5 wt % to about 9.0 wt %, from about 9.0 wt % to about 9.5 wt %, from about 9.5 wt % to about 10.0 wt %, or greater than about 10.0 wt % including all subranges in-between. In one embodiment, the granules of dimagnesium malate have a water content from at least 2.0 wt % to about 10.0 wt %. In yet another non-limiting embodiment, the granules of dimagnesium malate have a water content of up to about 30.0 wt %.

In one or more embodiments, the water content in the granules of dimagnesium malate may range from 2 wt % to 3 wt %, 2 wt % to 4 wt %, 2 wt % to 5 wt %, 2 wt % to 6 wt %, 2 wt % to 7 wt %, 2 wt % to 8 wt %, 2 wt % to 9 wt %, 2 wt % to 10 wt %, 3 wt % to 10 wt %, 4 wt % to 10 wt %, 5 wt % to 10 wt %, 6 wt % to 10 wt %, 7 wt % to 10 wt %, 8 wt % to 10 wt %, or 9 wt % to 10 wt %. In yet other embodiments, the granules of dimagnesium malate may range from 3 wt % to 9 wt %, 4 wt % to 9 wt %, 5 wt % to 9 wt %, 6 wt % to 9 wt %, 7 wt % to 9 wt %, or 8 wt % to 9 wt %.

In general, the compositions comprising granules of dimagnesium malate have a magnesium content (magnesium percentage, Mg %) of at least 18 wt % or greater. In various embodiments, the granules of dimagnesium malate have a magnesium content of at least 18 wt %, at least 19 wt %, at least 20 wt %, at least 21 wt %, at least 22 wt %, at least 23 wt %, at least 24 wt %, at least 25 wt %, at least 26 wt %, at least 27 wt %, at least 28 wt %, at least 29 wt %, at least 30 wt %, at least 31 wt %, at least 32 wt %, at least 33 wt %, at least 34 wt %, at least 35 wt %, at least 36 wt %, at least 37 wt %, at least 38 wt %, at least 39 wt %, at least 40 wt %, at least 41 wt %, at least 42 wt %, at least 43 wt %, at least 44 wt %, or at least 45 wt %, at least 46 wt %, at least 47 wt %, at least 48 wt %, at least 49 wt %, or about 50 wt %. In an embodiment, the compositions comprising granules of dimagnesium malate have a magnesium content (Mg %) of at least 18 wt % to about 30 wt %.

The compositions comprising granules of dimagnesium malate have a particle size ranging from about 50 microns to about 1500 microns. In various embodiments, the compositions comprising granules of dimagnesium malate have a particle size ranging from about 50 microns to about 1500 microns, from about 150 microns to about 1200 microns, from about 500 microns to about 1000 microns, or from about 700 microns to about 850 microns, including all subranges in-between. In one embodiment, the compositions comprising granules of dimagnesium malate where at least 75 wt % of the granules of dimagnesium malate have a particle size ranging from about 90 microns to about 1000 microns including all subranges in-between.

In one or more embodiments, the granules of dimagnesium malate may have a particle size ranging from 50 microns to 100 microns, from 50 microns to 200, from 50 microns to 300 microns, from 50 microns to 400 microns, from 50 microns to 500 microns, from 50 microns to 600, from 50 microns to 700 microns, from 50 microns to 800 microns, from 50 microns to 900 microns, from 50 microns to 1000, from 50 microns to 1100 microns, from 50 microns to 1200 microns, from 50 microns to 1300 microns, from 50 microns to 1400 microns, from 50 microns to 1500 microns, from 100 microns to 1500, from 200 microns to 1500 microns, from 300 microns to 1500 microns, from 400 microns to 1500 microns, from 500 microns to 1500, from 600 microns to 1500 microns, from 700 microns to 1500 microns, from 800 microns to 1500 microns, from 900 microns to 1500, from 1000 microns to 1500 microns, from 1100 microns to 1500 microns, from 1200 microns to 1500 microns, from 1300 microns to 1500 microns, or from 1400 microns to 1500 microns. In yet other embodiments, the granules of dimagnesium malate may have a particle size ranging from 500 microns to 1000 microns, 600 microns to 900 microns, or 700 microns to 800 microns.

In a preferred embodiment, 85 wt % to 95 wt % of the granules of dimagnesium malate may have a particle size ranging from about 90 microns to about 1000 microns.

Generally, the compositions comprising granules of dimagnesium malate have a bulk density of at least 0.70 g/mL. In various embodiments the granules of dimagnesium malate have a bulk density of at least 0.70 g/mL, at least 0.71 g/mL, at least 0.72 g/mL, at least 0.73 g/mL, at least 0.74 g/mL, at least 0.75 g/mL, at least 0.76 g/mL, at least 0.77 g/mL, at least 0.78 g/mL, at least 0.79 g/mL, at least 0.80 g/mL, at least 0.81 g/mL, at least 0.82 g/mL, at least 0.83 g/mL, at least 0.84 g/mL, at least 0.85 g/mL, at least 0.86 g/mL, at least 0.87 g/mL, at least 0.88 g/mL, at least 0.89 g/mL, at least 0.90 g/mL, at least 0.91 g/mL, at least 0.92 g/mL, at least 0.93 g/mL, at least 0.94 g/mL, at least 0.95 g/mL, at least 0.96 g/mL, at least 0.97 g/mL, at least 0.98 g/mL, at least 0.99 g/mL, at least 1.00 g/mL, at least 1.01 g/mL, at least 1.02 g/mL, at least 1.03 g/mL, at least 1.04 g/mL, at least 1.05 g/mL, at least 1.06 g/mL, at least 1.07 g/mL, at least 1.08 g/mL, at least 1.09 g/mL, at least 1.10 g/mL, or at least 1.10 g/mL or greater.

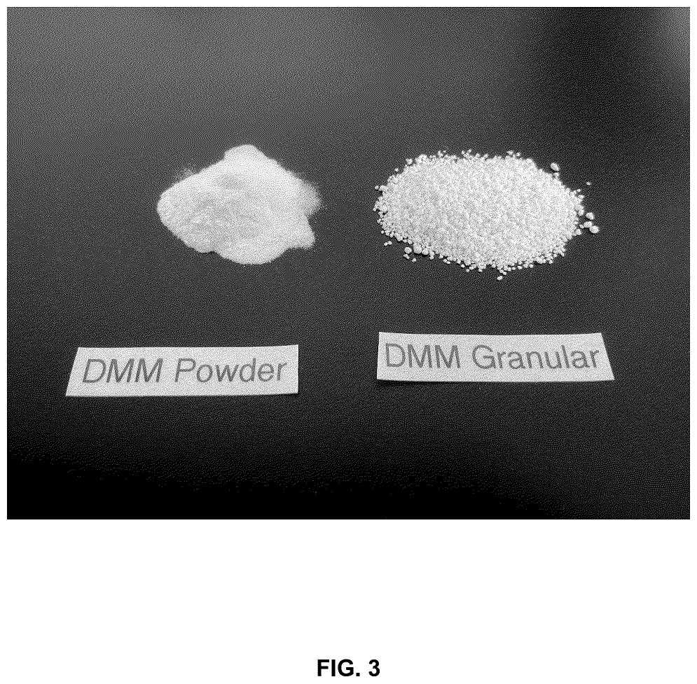

FIG. 3 shows a photographic comparison of the granules of dimagnesium malate (right) from the reactive granulation as compared to powders prepared through other conventional methods (left).

The composition comprising granules of dimagnesium malate may further comprise at least one pharmaceutically acceptable excipient. Examples of pharmaceutically acceptable excipients are detailed above in Section (I).

In some embodiments, the composition may comprise no more than 5.0 wt % silica. In some non-limiting embodiments, the composition may comprise no more than 5.0 wt % silica, no more than 4.5 wt % silica, no more than 4.0 wt % silica, no more than 3.5 wt % silica, no more than 3.0 wt % silica, no more than 2.5 wt % silica, no more than 2.0 wt % silica, no more than 1.5 wt % silica, no more than 1.0 wt % silica, no more than 0.5 wt % silica, no more than 0.25 wt % silica, no more than 0.1 wt % silica, no more than 0.09 wt % silica, no more than 0.08 wt % silica, no more than 0.07 wt % silica, no more than 0.06 wt % silica, no more than 0.05 wt % silica, no more than 0.04 wt % silica, no more than 0.03 wt % silica, no more than 0.02 wt % silica, no more than 0.01 wt % silica, or no more than 0.001 wt % silica. In some additional non-limiting embodiments, the composition may be free of silica.

### (IV) Methods for Preparing Granules of Calcium Citrate Malate

The present disclosure also relates to methods for preparing granules of calcium citrate malate. The methods comprise the following steps: (a) contacting citric acid and malic acid forming a particulate mixture; (b) adding water to the particulate mixture from step (a) forming a slurry; (c) contacting a calcium base with the slurry from step (b); (d) reacting to form the granules of calcium citrate malate; and (e) isolating the granules of calcium citrate malate; wherein the granules of calcium citrate malate have a water content of at least 5.0 wt % or greater and do not contain more than about 5.0 wt % of silica. In one or more embodiments, the produced granules of calcium citrate malate do not contain silica. The granules of calcium citrate malate have a mole ratio of calcium:malate:citrate of about 6:3:2. The methods for preparing granules of calcium citrate malate may be conducted in a batch mode or a semi-continuous mode.

The method, as detailed below, may be performed under ambient pressure. The method may also be conducted under an inert atmosphere, for example, under helium nitrogen, argon, or a combination thereof.

(a) Contacting Citric Acid and Malic Acid Forming a Particulate Mixture

The first step comprises contacting citric acid and malic acid forming a particulate mixture. Malic acid may be DL-malic acid, L-malic acid, or D-malic acid.

The citric acid and the malic acid are both in the form of solids such as a powder. Citric acid and malic acid may be added in any sequential order, in any combination, or all at once. Once added, the solids may be mixed according to known methods in the art such as a blender to achieve a well dispersed particulate mixture.

The temperature of contacting citric acid and malic acid forming a particulate mixture in step (a) may range from about 0° C. to about 50° C. In various embodiments, the temperature of contacting citric acid and malic acid forming a particulate mixture in step (a) ranges from about 0° C. to about 50° C., from about 10° C. to about 35° C., or from about 20° C. to about 30° C. In an embodiment, the temperature of contacting citric acid and malic acid forming a particulate mixture in step (a) is about 23° C. (room temperature).

In one or more embodiments, the temperature of contacting citric acid and malic acid forming a particulate mixture in step (a) may range from 0° C. to 5° C., from 0° C. to 10° C., from 0° C. to 15° C., from 0° C. to 20° C., from 0° C. to 25° C., from 0° C. to 30° C., from 0° C. to 35° C., from 0° C. to 40° C., from 0° C. to 45° C., from 0° C. to 50° C., from 5° C. to 50° C., from 10° C. to 50° C., from 15° C. to 50° C., from 20° C. to 50° C., from 25° C. to 50° C., from 30° C. to 50° C., from 35° C. to 50° C., from 40° C. to 50° C., or from 45° C. to 50° C. In yet other embodiments, the temperature of contacting citric acid and malic acid forming a particulate mixture in step (a) may range from 10° C. to 40° C. or from 20° C. to 30° C.

The citric acid and malic acid may be mixed. The duration of mixing in step (a) may range from about 1 minutes to about 30 minutes until a well dispersed particulate mixture is achieved. In various embodiments, the duration of contacting citric acid and malic acid forming a well dispersed particulate mixture ranges from about 1 minutes to about 30 minutes, from about 2 minutes to about 20 minutes, or from about 3 minutes to about 10 minutes until a particulate mixture is obtained. The determination of the completion of the particulate mixture may be done visually.

In one or more embodiments, the duration of mixing in step (a) may range from 1 minute to 5 minutes, from 1 minute to 10 minutes, from 1 minute to 15 minutes, from 1 minute to 20 minutes, from 1 minute to 25 minutes, from 1 minute to 30 minutes, 5 minutes to 30 minutes, 10 minutes to 30 minutes, 5 minutes to 30 minutes, 10 minutes to 30 minutes, 15 minutes to 30 minutes, 20 minutes to 30 minutes, or 25 minutes to 30 minutes. In yet other embodiments, the duration of mixing in step (a) may range from 5 minutes to 25 minutes, 10 minutes to 20 minutes, or 10 minutes to 15 minutes.

(b) Adding Water to the Particulate Mixture from Step (a) Forming a Slurry

The next step in the method comprises adding water to the particulate mixture from step (a) forming a slurry. The water may be deionized or distilled.

Generally, the total weight to volume ratio of the citric acid and the malic acid to the water may range from about 20.0:1.0 to about 100.0:1.0. In various embodiments, the total weight to volume ratio of the citric acid and the malic acid to the water may range from 20.0:1.0 to 100.0:1.0, from about 25.0:1.0 to about 75.0:1.0, or from about 30.0:1.0 to about 60.0:1.0. In one embodiment, the total weight to volume ratio of the citric acid and the malic acid to the water may range from about 45.0:1.0 to about 65.0.0:1.0.

The temperature of adding water to the particulate mixture from step (a) forming a slurry may range from about 0° C. to about 50° C. In various embodiments, the temperature of adding water to the particulate mixture from step (a) forming a slurry ranges from about 0° C. to about 50° C., from about 10° C. to about 35° C., or from about 20° C. to about 30° C. In an embodiment, the temperature in step (b) is about 23° C. (room temperature).

In one or more embodiments, the temperature of adding water to the particulate mixture from step (a) forming a slurry may range from 0° C. to 5° C., from 0° C. to 10° C., from 0° C. to 15° C., from 0° C. to 20° C., from 0° C. to 25° C., from 0° C. to 30° C., from 0° C. to 35° C., from 0° C. to 40° C., from 0° C. to 45° C., from 0° C. to 50° C., from 5° C. to 50° C., from 10° C. to 50° C., from 15° C. to 50° C., from 20° C. to 50° C., from 25° C. to 50° C., from 30° C. to 50° C., from 35° C. to 50° C., from 40° C. to 50° C., or from 45° C. to 50° C. In yet other embodiments, the temperature of adding water to the particulate mixture from step (a) forming a slurry may range from 10° C. to 40° C. or from 20° C. to 30° C.

The water and the particulate mixture from step (a) may be mixed. The duration of mixing in step (b) may range from about 1 minutes to about 30 minutes until a slurry is obtained. In various embodiments, the duration of mixing in step (b) may range from about 1 minutes to about 30 minutes, from about 5 minutes to about 25 minutes, or from about 10 minutes to about 20 minutes until a slurry is obtained. The determination of the completion of the slurry may be done visually.

In one or more embodiments, the duration of mixing in step (b) may range from 1 minute to 5 minutes, from 1 minute to 10 minutes, from 1 minute to 15 minutes, from 1 minute to 20 minutes, from 1 minute to 25 minutes, from 1 minute to 30 minutes, 5 minutes to 30 minutes, 10 minutes to 30 minutes, 5 minutes to 30 minutes, 10 minutes to 30 minutes, 15 minutes to 30 minutes, 20 minutes to 30 minutes, or 25 minutes to 30 minutes. In yet other embodiments, the duration of mixing in step (b) may range from 5 minutes to 25 minutes, 10 minutes to 20 minutes, or 10 minutes to 15 minutes.

(c) Contacting a Calcium Base with the Slurry from Step (b)

The next step in the method comprises contacting a calcium base with the slurry from step (b).

A wide variety of calcium bases may be used in step (c) of the method. Generally, these calcium bases possess high purity. In order to produce the granules of calcium citrate malate, the calcium base cannot be formed in-situ. Non-limiting examples of the calcium bases may be calcium carbonate, calcium oxide, calcium hydroxide, calcium metal, or a combination thereof. In one embodiment, the useful calcium base is calcium hydroxide.

As appreciated by one skilled in the art, contacting the calcium base with an acid produces an exotherm. In order to control the exotherm, the calcium base may be slowly added (metered), may be added in portions, the reactor may be cooled, or a combination thereof. Using a metered addition or portioned addition, the exotherm is controlled and the exotherm is allowed to dissipate before the next addition of the calcium base.

The calcium base may be added (metered) in portions to the slurry containing the citric acid and malic acid or slowly added continuously to the slurry containing the citric acid and malic acid. In one embodiment, the calcium base is added in two portions, each portion of similar weight or each portion having different weights. In another embodiment, the calcium base is added in more than two portions, each portion having a similar weight or different weights. In yet another embodiment, the calcium base is slowly added continuously. Using these addition sequences, the exotherm may be monitored and controlled.

The temperature of contacting the calcium base with the slurry from step (b) may range from about 0° C. to about 50° C. In various embodiments, the temperature of contacting calcium base with the slurry from step (c) may range from about 0° C. to about 50° C., from about 10° C. to about 35° C., or from about 20° C. to about 30° C. In an embodiment, the temperature of contacting the calcium base with the slurry from step (c) is about 23° C. (room temperature).

In one or more embodiments, the temperature in step (b) may range from 0° C. to 5° C., from 0° C. to 10° C., from 0° C. to 15° C., from 0° C. to 20° C., from 0° C. to 25° C., from 0° C. to 30° C., from 0° C. to 35° C., from 0° C. to 40° C., from 0° C. to 45° C., from 0° C. to 50° C., from 5° C. to 50° C., from 10° C. to 50° C., from 15° C. to 50° C., from 20° C. to 50° C., from 25° C. to 50° C., from 30° C. to 50° C., from 35° C. to 50° C., from 40° C. to 50° C., or from 45° C. to 50° C. In yet other embodiments, the temperature of contacting citric acid and malic acid forming a particulate mixture in step (a) may range from 10° C. to 40° C. or from 20° C. to 30° C.

As the calcium base contacts the slurry from step (b), an exotherm occurs. Thus, the rate at which the calcium base may be added controls the exotherm. The maximum exotherm temperature of this method step may range from about 70° C. to about 100° C. In various embodiments, the maximum exotherm temperature of this addition may range from about 70° C. to about 100° C., from about 70° C. to about 75° C., from about 75° C. to about 80° C., from about 80° C. to about 85° C., from about 85° C. to about 90° C., from about 90° C. to about 95° C., or from about 95° C. to about 100° C. In one embodiment, the maximum exotherm temperature of this method step ranges from about 85° C. to about 90° C.

In one or more embodiments, the maximum exotherm temperature of this method step may range from 70° C. to 75° C., from 70° C. to 80° C., from 70° C. to 85° C., from 70° C. to 90° C., from 70° C. to 95° C., from 70° C. to 100° C., from 75° C. to 100° C., from 80° C. to 100° C., from 85° C. to 100° C., from 90° C. to 100° C., or from 95° C. to 100° C., In yet other embodiments the maximum exotherm temperature of this method step may range from 75° C. to 95° C., from 80° C. to 90° C., or from 85° C. to 90° C.

The calcium base and the slurry from step (b) may be mixed. The duration of mixing in step (c) can and will vary depending on the batch size, the mixing apparatus used, the addition rate of the calcium base, and the maximum exotherm achieved after the addition. The duration of mixing in step (c) may range from about 5 minutes to about 120 minutes until a slurry is obtained. In various embodiments, the duration of mixing in step (c) may range from about 5 minutes to about 120 minutes, from about 30 minutes to about 100 minutes, or from about 60 minutes to about 90 minutes until a slurry is obtained.

In one or more embodiments, the duration of mixing in step (c) may range from 5 minutes to 10 minutes, from 5 minutes to 15 minutes, from 5 minutes to 20 minutes, from 5 minutes to 25 minutes, 5 minutes to 30 minutes, from 5 minutes to 35 minutes, from 5 minutes to 40 minutes, from 5 minutes to 45 minutes, 5 minutes to 50 minutes, from 5 minutes to 55 minutes, from 5 minutes to 60 minutes, from 5 minutes to 65 minutes, 5 minutes to 70 minutes, from 5 minutes to 75 minutes, from 5 minutes to 80 minutes, from 5 minutes to 90 minutes, from 5 minutes to 95 minutes, from 5 minutes to 100 minutes, 5 minutes to 105 minutes, from 5 minutes to 110 minutes, from 5 minutes to 115 minutes, from 5 minutes to 120 minutes, from 10 minutes to 120 minutes, from 15 minutes to 120 minutes, from 20 minutes to 120 minutes, from 25 minutes to 120 minutes, from 30 minutes to 120 minutes, from 35 minutes to 120 minutes, from 40 minutes to 120 minutes, from 45 minutes to 120 minutes, from 50 minutes to 120 minutes, from 55 minutes to 120 minutes, from 60 minutes to 120 minutes, from 65 minutes to 120 minutes, from 70 minutes to 120 minutes, from 75 minutes to 120 minutes, from 80 minutes to 120 minutes, from 85 minutes to 120 minutes, from 90 minutes to 120 minutes, from 95 minutes to 120 minutes, from 100 minutes to 120 minutes, from 105 minutes to 120 minutes, from 110 minutes to 120 minutes, or from 115 minutes to 120 minutes. In yet other embodiments, the duration of step (b) may range from 10 minutes to 115 minutes, 15 minutes to 110 minutes, 20 minutes to 105 minutes, 25 minutes to 100 minutes, 30 minutes to 95 minutes, 35 minutes to 90 minutes, 40 minutes to 85 minutes, 45 minutes to 80 minutes, 50 minutes to 75 minutes, 55 minutes to 70 minutes, or 60 minutes to 65 minutes.

(d) Reacting to Form the Granules of Calcium Citrate Malate

The next step in the method, step (d), reacting to form the granules of calcium citrate malate. Upon complete addition of the calcium base to the particulate mixture of citric acid and malic acid, the granules of calcium citrate malate essentially form. Additional time may be needed to completely form the granules.

(e) Isolating the Granules of Calcium Citrate Malate

The last step in the process, step (e), comprises isolating the granules of calcium citrate malate. Generally, the granules of calcium citrate malate are simply removed from the reaction vessel. The granules, after isolation, may be further dried.

After drying, the granules of calcium citrate malate may have a yield of at least about 75%. In various embodiments, the granules of calcium citrate malate may have a yield of at least about 75%, at least 80%, or at least about 90%.

The isolated granules of calcium citrate malate have a total aerobic count less than 1000 CFU/g. In various embodiments, the granules of calcium citrate malate may have a total aerobic count less than about 900 CFU/g, less than about 800 CFU/g, less than about 700 CFU/g, less than about 600 CFU/g, less than about 500 CFU/g, less than about 400 CFU/g, less than about 300 CFU/g, less than about 200 CFU/g, or less than about 100 CFU/g.

### (V) Methods for Preparing Granules of Dicalcium Malate

The present disclosure also relates to methods for preparing granules of dicalcium malate. The method comprises the following steps: (a) contacting a calcium base with water forming a slurry; (b) contacting the slurry from step (a) with malic acid causing a reaction between the malic acid and the calcium base forming a second slurry; (c) repeating step (b) until the calcium base and malic acid react forming granules of dicalcium malate; and (d) isolating the granules of dicalcium malate; wherein the granules of dicalcium malate have a water content of at least 5.0 wt % or greater and do not contain more than about 5.0 wt % of silica. In one or more embodiments, the produced granules of dicalcium malate do not contain silica. The granules of dicalcium malate may have a mole ratio of calcium to malate of about 2:1. The methods for preparing granules of dicalcium malate may be conducted in a batch mode or a semi-continuous mode.

The method, as detailed below, may be performed under ambient pressure. The method may also be conducted under an inert atmosphere, for example, under helium nitrogen, argon, or a combination thereof.

(a) Contacting a Calcium Base with Water Forming a Slurry

The first step in the method, step (a), comprises contacting a calcium base with water forming a slurry. Various calcium bases useful in this method are described in more detail above in Section (IV). In one embodiment, the calcium base useful in this method is calcium hydroxide.

Generally, the total weight to volume ratio of the calcium base to the water may range from 20.0:1.0 to 100.0:1.0. In various embodiments, the total weight to volume ratio of the calcium base to the water may range from 20.0:1.0 to 100.0:1, from about 30.0:1.0 to about 80.0:1.0, or from about 50.0:1.0 to about 75.0:1.0. In one embodiment, the weight to volume ratio of the calcium base to the water may range from 55.0:1.0 to 75.0:1.0.

The temperature of contacting a calcium base with water forming a slurry may range from about 0° C. to about 50° C. In various embodiments, the temperature in step (a) ranges from about 0° C. to about 50° C., from about 10° C. to about 35° C., or from about 20° C. to about 30° C. In an embodiment, the temperature of contacting a calcium base with water forming a slurry is about 23° C. (room temperature).

In one or more embodiments, the temperature of contacting a calcium base with water forming a slurry may range from 0° C. to 5° C., from 0° C. to 10° C., from 0° C. to 15° C., from 0° C. to 20° C., from 0° C. to 25° C., from 0° C. to 30° C., from 0° C. to 35° C., from 0° C. to 40° C., from 0° C. to 45° C., from 0° C. to 50° C., from 5° C. to 50° C., from 10° C. to 50° C., from 15° C. to 50° C., from 20° C. to 50° C., from 25° C. to 50° C., from 30° C. to 50° C., from 35° C. to 50° C., from 40° C. to 50° C., or from 45° C. to 50° C. In yet other embodiments, the temperature of contacting water and calcium base forming a particulate mixture of contacting a calcium base with water forming a slurry may range from 10° C. to 40° C. or from 20° C. to 30° C.

The calcium base and water may be mixed. The duration of mixing in step (a) may range from about 1 minutes to about 30 minutes until a slurry is obtained. In various embodiments, the duration of mixing in step (a) may range from about 1 minutes to about 30 minutes, from about 5 minutes to about 25 minutes, or from about 10 minutes to about 20 minutes until a slurry is obtained. The determination of the completion of the slurry may be done visually.

In one or more embodiments, the duration of mixing in step (a) may range from 1 minute to 5 minutes, from 1 minute to 10 minutes, from 1 minute to 15 minutes, from 1 minute to 20 minutes, from 1 minute to 25 minutes, from 1 minute to 30 minutes, 5 minutes to 30 minutes, 10 minutes to 30 minutes, 5 minutes to 30 minutes, 10 minutes to 30 minutes, 15 minutes to 30 minutes, 20 minutes to 30 minutes, or 25 minutes to 30 minutes. In yet other embodiments, the duration of mixing in step (a) may range from 5 minutes to 25 minutes, 10 minutes to 20 minutes, or 10 minutes to 15 minutes.

(b) Contacting the Slurry from Step (a) with Malic Acid Causing a Reaction Between the Malic Acid and the Calcium Base Forming a Second Slurry

The next step in the method, step (b), comprises contacting the slurry from step (a) with malic acid causing a reaction between the malic acid and the calcium base to react forming a second slurry. The malic acid may be DL-malic acid, L-malic acid, or D-malic acid.

The malic acid may be added in two, three, or more than three portions to the calcium base. The portions of malic acid may be of equal weight or different weights. The malic acid may also be slowly added continuously to the calcium base.

As the malic acid contacts the calcium base, an exotherm occurs. Thus, the rate at which the malic acid is added may control the exotherm. The maximum exotherm temperature of this method step may range from about 70° C. to about 100° C. In various embodiments, the maximum exotherm temperature of the method step may range from about 70° C. to about 100° C., from about 70° C. to about 75° C., from about 75° C. to about 80° C., from about 80° C. to about 85° C., from about 85° C. to about 90° C., from about 90° C. to about 95° C., or from about 95° C. to about 100° C. In one embodiment, the maximum exotherm temperature of this method step ranges from about 80° C. to about 90° C.

In one or more embodiments, the maximum exotherm temperature of this method step may range from 70° C. to 75° C., from 70° C. to 80° C., from 70° C. to 85° C., from 70° C. to 90° C., from 70° C. to 95° C., from 70° C. to 100° C., from 75° C. to 100° C., from 80° C. to 100° C., from 85° C. to 100° C., from 90° C. to 100° C., or from 95° C. to 100° C., In yet other embodiments the maximum exotherm temperature of this method step may range from 75° C. to 95° C., from 80° C. to 90° C., or from 85° C. to 90° C.

The malic acid and the slurry from step (a) may be mixed. The duration of mixing in step (b) can and will vary depending on the batch size, the mixing apparatus used, the addition rate of the calcium base, whether the malic acid is added in portions or continuously, and the maximum exotherm achieved. The duration of step (b) may range from about 5 minutes to about 120 minutes until a slurry is obtained. In various embodiments, the duration of mixing in step (b) may range from about 5 minutes to about 120 minutes, from about 30 minutes to about 90 minutes, or from about 45 minutes to about 60 minutes until a slurry is obtained.

In one or more embodiments, the duration of mixing in step (b) may range from 5 minutes to 10 minutes, from 5 minutes to 15 minutes, from 5 minutes to 20 minutes, from 5 minutes to 25 minutes, 5 minutes to 30 minutes, from 5 minutes to 35 minutes, from 5 minutes to 40 minutes, from 5 minutes to 45 minutes, 5 minutes to 50 minutes, from 5 minutes to 55 minutes, from 5 minutes to 60 minutes, from 5 minutes to 65 minutes, 5 minutes to 70 minutes, from 5 minutes to 75 minutes, from 5 minutes to 80 minutes, from 5 minutes to 90 minutes, from 5 minutes to 95 minutes, from 5 minutes to 100 minutes, 5 minutes to 105 minutes, from 5 minutes to 110 minutes, from 5 minutes to 115 minutes, from 5 minutes to 120 minutes, from 10 minutes to 120 minutes, from 15 minutes to 120 minutes, from 20 minutes to 120 minutes, from 25 minutes to 120 minutes, from 30 minutes to 120 minutes, from 35 minutes to 120 minutes, from 40 minutes to 120 minutes, from 45 minutes to 120 minutes, from 50 minutes to 120 minutes, from 55 minutes to 120 minutes, from 60 minutes to 120 minutes, from 65 minutes to 120 minutes, from 70 minutes to 120 minutes, from 75 minutes to 120 minutes, from 80 minutes to 120 minutes, from 85 minutes to 120 minutes, from 90 minutes to 120 minutes, from 95 minutes to 120 minutes, from 100 minutes to 120 minutes, from 105 minutes to 120 minutes, from 110 minutes to 120 minutes, or from 115 minutes to 120 minutes. In yet other embodiments, the duration of mixing in step (b) may range from 10 minutes to 115 minutes, 15 minutes to 110 minutes, 20 minutes to 105 minutes, 25 minutes to 100 minutes, 30 minutes to 95 minutes, 35 minutes to 90 minutes, 40 minutes to 85 minutes, 45 minutes to 80 minutes, 50 minutes to 75 minutes, 55 minutes to 70 minutes, or 60 minutes to 65 minutes.

(c) Repeating Step (b) Until the Calcium Base and Malic Acid Fully React Forming Granules of Dicalcium Malate

The next step of the method, step (c), additional portion of malic acid are added to convert the remaining calcium base into dicalcium malate. The malic acid may be added in two portions, three portions, four portions, or more than four portions, each portion may be of equal of equal weight or unequal weight. In one embodiment, the malic acid may be added in two equal portions. In another embodiment, the malic acid may be added in two unequal portions. In still another embodiment, the malic acid may be added in three equal portions. In yet another embodiment, the malic acid is added in three unequal portions. In still another embodiment, the malic acid may be added continuously.

In each addition of malic acid, the maximum exotherm temperature may range from about 70° C. to about 100° C. In various embodiments, the maximum exotherm temperature may range from about 70° C. to about 100° C., from about 70° C. to about 75° C., from about 75° C. to about 80° C., from about 80° C. to about 85° C., from about 85° C. to about 90° C., from about 90° C. to about 95° C., or from about 95° C. to about 100° C. In one embodiment, the maximum exotherm temperature of this step ranges from about 80° C. to about 90° C. Once this exotherm dissipates, the reaction is considered complete.

In one or more embodiments, the maximum exotherm temperature of this method step may range from 70° C. to 75° C., from 70° C. to 80° C., from 70° C. to 85° C., from 70° C. to 90° C., from 70° C. to 95° C., from 70° C. to 100° C., from 75° C. to 100° C., from 80° C. to 100° C., from 85° C. to 100° C., from 90° C. to 100° C., or from 95° C. to 100° C., In yet other embodiments the maximum exotherm temperature of this method step may range from 75° C. to 95° C., from 80° C. to 90° C., or from 85° C. to 90° C.

(d) Isolating the Granules of Dicalcium Malate

The last step in the method, step (e), comprises isolating the dicalcium malate. Generally, the granules of dicalcium malate are simply removed from the reaction vessel. The granules, after isolation, may be further dried and milled.

After drying, the granules of dicalcium malate may have a yield of at least about 75%. In various embodiments, the granules of dicalcium malate may have a yield of at least about 75%, at least 80%, or at least about 90%.

The isolated granules of dicalcium malate have a total aerobic count less than 1000 CFU/g. In various embodiments, the granules of dicalcium malate may have a total aerobic count less than about 900 CFU/g, less than about 800 CFU/g, less than about 700 CFU/g, less than about 600 CFU/g, less than about 500 CFU/g, less than about 400 CFU/g, less than about 300 CFU/g, less than about 200 CFU/g, or less than about 100 CFU/g.

### (VI) Methods for Preparing Granules of Dimagnesium Malate

The present disclosure also relates to methods for preparing granules of dimagnesium malate. The method comprises the following steps: (a) contacting the magnesium base and the malic acid forming a particulate mixture; (b) contacting the particulate mixture from step (a) with water causing a reaction between the malic acid and the magnesium base forming a mixture; (c) repeating step (b) until the malic acid and magnesium base are fully reacted forming granules; and (d) isolating the granules of dimagnesium malate; wherein the granules of dimagnesium malate have a water content of at least 2.0 wt % or greater and do not contain more than about 5.0 wt % of silica. In one or more embodiments, the produced granules of dimagnesium malate do not contain silica. The granules of dimagnesium malate may have a mole ratio of magnesium to malate of about 2:1. The methods for preparing granules of dimagnesium malate may be conducted in a batch mode or a semi-continuous mode.

The method, as detailed below, may be performed under ambient pressure. The method may also be conducted under an inert atmosphere, for example, under helium nitrogen, argon, or a combination thereof.

(a) Contacting the Magnesium Base and Malic Acid Forming a Particulate Mixture

The first step in the method comprises contacting magnesium base and malic acid forming a particulate mixture.

A wide variety of magnesium bases may be used in step (a) of the method. Generally, these magnesium bases possess have high purity. In order to produce the granules of magnesium malate, the magnesium base cannot be formed in-situ. Non-limiting examples of the magnesium bases may be magnesium carbonate, magnesium oxide, magnesium hydroxide, or a combination thereof. In one embodiment, the useful magnesium base is magnesium oxide.

The malic acid may be DL-malic acid, L-malic acid, or D-malic acid.

The magnesium base and the malic acid are both in the form of solids such as a powder. The magnesium base and malic acid may be added in any sequential order, in any combination, or all at once. Once added, the solids may be mixed according to known methods in the art such as a blender.

The temperature of contacting magnesium base and malic acid forming a particulate mixture in step (a) may range from about 0° C. to about 50° C. In various embodiments, the temperature of contacting magnesium base and malic acid forming a particulate mixture in step (a) ranges from about 0° C. to about 50° C., from about 10° C. to about 35° C., or from about 20° C. to about 30° C. In an embodiment, the temperature of contacting magnesium base and malic acid forming a particulate mixture in step (a) is about 23° C. (room temperature).

In one or more embodiments, the temperature of contacting magnesium base and malic acid forming a particulate mixture in step (a) may range from 0° C. to 5° C., from 0° C. to 10° C., from 0° C. to 15° C., from 0° C. to 20° C., from 0° C. to 25° C., from 0° C. to 30° C., from 0° C. to 35° C., from 0° C. to 40° C., from 0° C. to 45° C., from 0° C. to 50° C., from 5° C. to 50° C., from 10° C. to 50° C., from 15° C. to 50° C., from 20° C. to 50° C., from 25° C. to 50° C., from 30° C. to 50° C., from 35° C. to 50° C., from 40° C. to 50° C., or from 45° C. to 50° C. In yet other embodiments, the temperature of contacting citric acid and malic acid forming a particulate mixture in step (a) may range from 10° C. to 40° C. or from 20° C. to 30° C.

The magnesium base and malic acid may be mixed. The duration of mixing in step (a) may range from about 1 minutes to about 30 minutes until a particulate mixture is obtained. In various embodiments, the duration of contacting magnesium base and malic acid forming a particulate mixture ranges from about 1 minutes to about 30 minutes, from about 2 minutes to about 20 minutes, or from about 3 minutes to about 10 minutes until a particulate mixture is obtained. The determination of the completion of the particulate mixture may be done visually.

In one or more embodiments, the duration of mixing in step (a) may range from 1 minute to 5 minutes, from 1 minute to 10 minutes, from 1 minute to 15 minutes, from 1 minute to 20 minutes, from 1 minute to 25 minutes, from 1 minute to 30 minutes, 5 minutes to 30 minutes, 10 minutes to 30 minutes, 5 minutes to 30 minutes, 10 minutes to 30 minutes, 15 minutes to 30 minutes, 20 minutes to 30 minutes, or 25 minutes to 30 minutes. In yet other embodiments, the duration mixing in step (a) may range from 5 minutes to 25 minutes, 10 minutes to 20 minutes, or 10 minutes to 15 minutes.

(b) Adding Water to the Particulate Mixture from Step (a) Causing a Partial Reaction Between the Malic Acid and the Magnesium Base Forming a Mixture

The next step in the method, step (b), comprises adding water to the particulate mixture from step (a) causing a reaction between the malic acid and the magnesium base forming a slurry. Once the total volume of water is added, the reaction between the magnesium base and malic acid is essentially complete. The water may be deionized or distilled.

The water added in this method step may be added in portions or continuously. By the addition of the water in portions to the particulate mixture, this addition allows this method step to partially react and control the exotherm of the reaction. The portions may be similar in volume or different in volume. In various embodiments, the water is added in two portions, three portions, four portions, five portions, six portions, or more than seven portions. In another embodiment, the water is continuously added (sprayed) into the particulate mixture.

Generally, the total weight of the magnesium base/malic acid to the total volume of water may range from about 20.0:1.0 to about 100.0:1.0. In various embodiments, the total weight of the magnesium base/malic acid to the volume of water may range from about 20.0:1.0 to about 100.0:1.0, from about 40.0:1.0 to about 90.0:1.0, or from about 65.0:1.0 to about 85.0:1.0. In one embodiment, the total volume to weight ratio of the water to the magnesium base/malic acid may range from about 70.0:1.0 to about 80.0:1.0.

The temperature of adding water to the particulate mixture from step (a) may range from about 0° C. to about 50° C. In various embodiments, the temperature of adding water to the particulate mixture from step (a) may range from about 0° C. to about 50° C., from about 10° C. to about 35° C., or from about 20° C. to about 30° C. In an embodiment, the temperature of contacting the water with the particulate mixture from step (a) is about 23° C. (room temperature).

In one or more embodiments, the temperature of adding water to the particulate mixture from step (a) may range from 0° C. to 5° C., from 0° C. to 10° C., from 0° C. to 15° C., from 0° C. to 20° C., from 0° C. to 25° C., from 0° C. to 30° C., from 0° C. to 35° C., from 0° C. to 40° C., from 0° C. to 45° C., from 0° C. to 50° C., from 5° C. to 50° C., from 10° C. to 50° C., from 15° C. to 50° C., from 20° C. to 50° C., from 25° C. to 50° C., from 30° C. to 50° C., from 35° C. to 50° C., from 40° C. to 50° C., or from 45° C. to 50° C. In yet other embodiments, the temperature of adding water to the particulate mixture from step (a) may range from 10° C. to 40° C., from 20° C. to 30° C.

As the water contacts the particulate mixture from step (a), the reaction between malic acid and the magnesium base occurs and an exotherm is evident. Thus, the rate at which the water is added to the particulate mixture may control the exotherm.

The maximum exotherm temperature of this method step may range from about 80° C. to about 110° C. In various embodiments, the maximum exotherm temperature of the method step may range from about 80° C. to about 110° C., from about 80° C. to about 85° C., from about 85° C. to about 90° C., from about 90° C. to about 95° C., or from about 95° C. to about 100° C., from about 105° C. to about 100° C., or from about 105° C. to about 110° C. In one embodiment, the maximum exotherm temperature of this method step ranges from about 80° C. to about 95° C.

In one or more embodiments, the maximum exotherm temperature of this method step may range from 80° C. to 85° C., from 80° C. to 90° C., from 80° C. to 100° C., from 80° C. to 105° C., from 80° C. to 110° C., from 85° C. to 110° C., from 90° C. to 110° C., from 95° C. to 110° C., from 100° C. to 110° C., or from 105° C. to 110° C. In yet other embodiments the maximum exotherm temperature of this method step may range from 85° C. to 105° C., from 90° C. to 100° C., or from 95° C. to 100° C.

The duration of adding water to the particulate mixture from step (a) can and will vary depending on the batch size, the mixing apparatus used, the addition rate of the water, whether the water is added in portions or continuously added, and the maximum exotherm achieved upon adding the water. The duration of adding water to the particulate mixture from step (a) may range from about 5 minutes to about 120 minutes until a slurry is obtained. In various embodiments, the duration of step (b) may range from about 5 minutes to about 120 minutes, from about 30 minutes to about 90 minutes, or from about 45 minutes to about 60 minutes until a slurry is obtained.

In one or more embodiments, the duration of adding water to the particulate mixture from step (a) may range from 5 minutes to 10 minutes, from 5 minutes to 15 minutes, from 5 minutes to 20 minutes, from 5 minutes to 25 minutes, 5 minutes to 30 minutes, from 5 minutes to 35 minutes, from 5 minutes to 40 minutes, from 5 minutes to 45 minutes, 5 minutes to 50 minutes, from 5 minutes to 55 minutes, from 5 minutes to 60 minutes, from 5 minutes to 65 minutes, 5 minutes to 70 minutes, from 5 minutes to 75 minutes, from 5 minutes to 80 minutes, from 5 minutes to 90 minutes, from 5 minutes to 95 minutes, from 5 minutes to 100 minutes, 5 minutes to 105 minutes, from 5 minutes to 110 minutes, from 5 minutes to 115 minutes, from 5 minutes to 120 minutes, from 10 minutes to 120 minutes, from 15 minutes to 120 minutes, from 20 minutes to 120 minutes, from 25 minutes to 120 minutes, from 30 minutes to 120 minutes, from 35 minutes to 120 minutes, from 40 minutes to 120 minutes, from 45 minutes to 120 minutes, from 50 minutes to 120 minutes, from 55 minutes to 120 minutes, from 60 minutes to 120 minutes, from 65 minutes to 120 minutes, from 70 minutes to 120 minutes, from 75 minutes to 120 minutes, from 80 minutes to 120 minutes, from 85 minutes to 120 minutes, from 90 minutes to 120 minutes, from 95 minutes to 120 minutes, from 100 minutes to 120 minutes, from 105 minutes to 120 minutes, from 110 minutes to 120 minutes, or from 115 minutes to 120 minutes. In yet other embodiments, the duration of step (b) may range from 10 minutes to 115 minutes, 15 minutes to 110 minutes, 20 minutes to 105 minutes, 25 minutes to 100 minutes, 30 minutes to 95 minutes, 35 minutes to 90 minutes, 40 minutes to 85 minutes, 45 minutes to 80 minutes, 50 minutes to 75 minutes, 55 minutes to 70 minutes, or 60 minutes to 65 minutes.

(c) Repeating Step (b) Until the Magnesium Base and Malic Acid are Fully Reacted to Form Granules of Dimagnesium Malate

In this step in the method, step (c), additional portions of water are added to convert the remaining magnesium base and malic acid into dimagnesium malate. The water may be added in portions to the particulate mixture of the magnesium base and the malic acid or continuously added.

In each addition of water, the maximum exotherm temperature of this method step may range from about 80° C. to about 110° C. In various embodiments, the maximum exotherm temperature of the method step may range from about 80° C. to about 110° C., from about 80° C. to about 85° C., from about 85° C. to about 90° C., from about 90° C. to about 95° C., or from about 95° C. to about 100° C., from about 105° C. to about 100° C., or from about 105° C. to about 110° C. In one embodiment, the maximum exotherm temperature of this method step ranges from about 80° C. to about 95° C.

In one or more embodiments, the maximum exotherm temperature of this method step may range from 80° C. to 85° C., from 80° C. to 90° C., from 80° C. to 100° C., from 80° C. to 105° C., from 80° C. to 110° C., from 85° C. to 110° C., from 90° C. to 110° C., from 95° C. to 110° C., from 100° C. to 110° C., or from 105° C. to 110° C. In yet other embodiments the maximum exotherm temperature of this method step may range from 85° C. to 105° C., from 90° C. to 100° C., or from 95° C. to 100° C.

(d) Isolating the Granules of Dimagnesium Malate

The last step in the method, step (e), comprises isolating the dimagnesium malate from the reaction mixture. Generally, the granules of dimagnesium malate are simply removed from the reaction vessel. The granules, after isolation, may be further dried and milled.

After drying, the granules of dimagnesium malate may have a yield of at least about 75%. In various embodiments, the granules of dimagnesium malate may have a yield of at least about 75%, at least 90%, or at least about 95%.

The isolated granules of dimagnesium malate have a total aerobic count less than about 1000 CFU/g. In various embodiments, the granules of dimagnesium malate may have a total aerobic count less than about 900 CFU/g, less than about 800 CFU/g, less than about 700 CFU/g, less than about 600 CFU/g, less than about 500 CFU/g, less than about 400 CFU/g, less than about 300 CFU/g, less than about 200 CFU/g, or less than about 100 CFU/g.

### (VII) Dosage Forms Comprising Compositions of Compressed Granules of Calcium Citrate Malate

The present disclosure provides dosage forms comprising compressed compositions of the calcium citrate malate described in Section I. The composition comprising granules of calcium citrate malate are described in more detail above in Section (I).

Dosage forms for oral administration may include capsules, tablets, caplets, pills, powders, pellets, and granules. In such dosage forms, the granules of calcium citrate malate may be combined with one or more pharmaceutically acceptable excipients, examples of which are detailed above. Oral preparations may also be administered as aqueous suspensions, elixirs, or syrups. In one embodiment, the dosage formulation comprising compressed granules of calcium citrate malate is a tablet.

### (VIII) Dosage Forms Comprising Compositions of Compressed Granules of Dicalcium Malate

The present disclosure provides dosage forms comprising compressed compositions of the dicalcium malate described in Section II. The composition comprising granules of dicalcium malate are described in more detail above in Section (II).

Dosage forms for oral administration may include capsules, tablets, caplets, pills, powders, pellets, and granules. In such dosage forms, the granules of dicalcium malate may be combined with one or more pharmaceutically acceptable excipients, examples of which are detailed above. Oral preparations may also be administered as aqueous suspensions, elixirs, or syrups. In one embodiment, the dosage formulation comprising compressed granules of dicalcium malate is a tablet.

### (VIII) Dosage Forms Comprising Compositions of Compressed Granules of Dimagnesium Malate

The present disclosure provides dosage forms comprising compressed compositions of the dimagnesium malate described in Section III. The composition comprising granules of dimagnesium malate are described in more detail above in Section (III).

Dosage forms for oral administration may include capsules, tablets, caplets, pills, powders, pellets, and granules. In such dosage forms, the granules of dimagnesium malate ordinarily combined with one or more pharmaceutically acceptable excipients, examples of which are detailed above. Oral preparations may also be administered as aqueous suspensions, elixirs, or syrups. In one embodiment, the dosage formulation comprising compressed granules of dimagnesium malate is a tablet.

### Definitions

When introducing elements of the embodiments described herein, the articles “a”, “an”, “the” and “said” are intended to mean that there are one or more of the elements. The terms “comprising”, “including” and “having” are intended to be inclusive and mean that there may be additional elements other than the listed elements.

As used herein, “about” indicates a quantity within 10% of the numerical value it precedes, consistent with the USP-NF definition of “about”. With regard to ratios (e.g., mol ratios), as used herein, a ratio of “about 2:1” would therefore refer to a ratio of 2±10%:1±10%.

As used herein, “water content” refers to the quantity of water contained in a material. Water content may be measured by direct or indirect methods. In direct methods, water is removed followed by measuring weight loss. In indirect methods, an intermediate variable is measured and then converted into water content. Water content may be measured by industry accepted methods. Non-limiting examples of how to measure water content include but are not limited to thermogravimetric analysis (TGA), oven, moisture balance, KF, and NIR.

As used herein, “water activity” is the thermodynamic activity of water as solvent and the relative humidity of the surrounding air after equilibration. High water activity generally refers to a water activity greater than 0.5 Aw.

As used herein, “calcium weight percentage” refers to the percent of calcium in a composition relative to the other elements. The amount of calcium may be measured by industry accepted methods. Non-limiting examples of how to measure the amount of calcium include but are not limited to ICP, ICPMS, FAAS, MPAES, XRF, or titration.

As used herein, “magnesium weight percentage” refers to the percent of magnesium in a composition relative to the other elements. The amount of magnesium may be measured by industry accepted methods. Non-limiting examples of how to measure the amount of magnesium include but are not limited to ICP, ICPMS, FAAS, MPAES, XRF, or titration.

As used herein, “bulk density” is the mass of the many particles of a material divided by the total volume occupied by the material. Bulk density may be measured by industry accepted methods. Non-limiting examples of how to measure bulk density include but are not limited to measurement in a graduated cylinder, measurement in a volumeter (with or without a baffle system), or measurement in a vessel.

As used herein, “total aerobic count” is a test that provides an indication of bacterial contamination in a sample. Methods of measuring total aerobic count are generally known in the art.

As various changes could be made in the above-described methods without departing from the scope of the invention, it is intended that all matter contained in the above description and in the examples given below, shall be interpreted as illustrative and not in a limiting sense.

### EXAMPLES

### Example 1: Preparation of Calcium Citrate Malate

Into a blender was added 315 lbs. of DL-malic acid and 300 lbs. of citric acid at room temperature. The blender was initiated, and the solid acids were blended for 30 minutes until the particulate mixture had formed. The particulate mixture was transferred into a reactor. Water (˜20 gal.) was added at room temperature to the reactor. After the water addition was complete, the reactor was stirred for 20 minutes until a slurry was obtained. 346 lbs. of calcium hydroxide were then added to the hopper and slowly added to the slurry. The addition rate of the calcium hydroxide was monitored so the internal temperature of the reactor did not exceed 90° C. After the calcium hydroxide addition was complete, the granules of calcium citrate malate were stirred for an additional 20 minutes. The granules of calcium citrate malate were removed from the reactor and dried for 24 hours under vacuum. The granules of calcium citrate malate were removed from the reactor and dried to a thermogravimetrically determined water content of 8.5 wt %. Total calcium content, determined by inductively coupled plasma analysis, was 20.3 wt %. 85% of the particle distribution was between 90 and 1000 microns determined by analytical sieving. The bulk density of the product was 0.86 g/mL.

### Example 2: Preparation of Dicalcium Malate

Into a reactor was added 250 lbs. of calcium hydroxide at room temperature. Water (˜20 gal.) was added at 35° C. to the reactor. After the water addition was complete, the reactor was stirred for 10 minutes until a slurry was obtained. 444 lbs. of DL-malic acid were then added to the hopper and slowly added to the slurry. The addition rate of the malic acid was monitored so the internal temperature of the reactor did not exceed 100° C. After the malic acid addition was complete, the granules of dicalcium malate were stirred for an additional 15 minutes. The granules of dicalcium malate were removed from the reactor and dried to a thermogravimetrically determined water content of 7.8 wt %. Total calcium content, determined by inductively coupled plasma analysis, was 29.3 wt %. 87% of the particle distribution was between 90 and 1000 microns determined by analytical sieving. The bulk density of the product was 0.77 g/mL.

### Example 3: Preparation of Dimagnesium Malate without Silica

Into a blender was added 331 lbs. of magnesium oxide and 550 lbs. of L-malic acid at room temperature. The blender was initiated, and the solid acids were blended for 5 minutes until the particulate mixture had formed. The particulate mixture was transferred into a reactor. Water (˜10 gal.) was added at room temperature to the reactor. The addition rate of the water was monitored so the internal temperature of the reactor did not exceed 110° C. After the water addition was complete, the granules of dimagnesium malate were stirred for an additional 45 minutes. The granules of dimagnesium malate were dried in the reactor to a thermogravimetrically determined water content of 3.3 wt %. Total magnesium content, determined by inductively coupled plasma analysis, was 20.5 wt %. 75% of the particle distribution was between 90 and 1000 microns determined by analytical sieving. The bulk density of the product was 0.98 g/mL.

### Example 4: Preparation of Dimagnesium Malate

Into a blender was added 331 lbs. of magnesium oxide and 550 lbs. of L-malic acid, and 10 lbs. of silica at room temperature. The blender was initiated, and the solid acids were blended for 5 minutes until the particulate mixture had formed. The particulate mixture was transferred into a reactor. Water (˜10 gal.) was added at room temperature to the reactor. The addition rate of the water was monitored so the internal temperature of the reactor did not exceed 110° C. After the water addition was complete, the granules of dimagnesium malate were stirred for an additional 45 minutes. The granules of dimagnesium malate were dried in the reactor to a thermogravimetrically determined water content of 3.8 wt %. Total magnesium content, determined by inductively coupled plasma analysis, was 21.1 wt %. 75% of the particle distribution was between 90 and 1000 microns determined by analytical sieving. The bulk density of the product was 0.98 g/mL

What is claimed is:

1. A composition comprising granules of calcium citrate malate having a mole ratio of calcium:malate:citrate of about 6:3:2 and a water content of at least 5.0 weight % (wt %).

2. The composition of claim 1, wherein the composition comprises at least one excipient.

3. The composition of claim 1, wherein the granules have a calcium weight percentage of at least 19 wt %.

4. The composition of claim 1, wherein the granules of calcium citrate malate have a particle size ranging from about 50 microns to about 1500 microns.

5. The composition of claim 1, wherein the granules have a bulk density of at least 0.70 g/mL.

6. The composition of claim 1, wherein the composition comprises no more than 5.0 wt % silica.

7. A composition comprising granules of dicalcium malate having a mole ratio of calcium:malate of about 2:1 and a water content of at least 5.0 wt %.

8. The composition of claim 7, wherein the composition comprises at least one excipient.

9. The composition of claim 7, wherein the granules have a calcium weight percentage of at least 27 wt %.

10. The composition of claim 7, wherein the granules of dicalcium malate have a particle size ranging from about 50 microns to about 1500 microns.

11. The composition of claim 7, wherein the granules have a bulk density of at least 0.70 g/mL.

12. The composition of claim 7, wherein the composition comprises no more than 5.0 wt % silica.

13. A composition comprising granules of dimagnesium malate having a mole ratio of magnesium:malate of about 2:1 and a water content of at least 2.0 wt %.

14. The composition of claim 13, wherein the composition comprises at least one excipient.

15. The composition of claim 13, wherein the granules have a magnesium weight percentage of at least 18 wt %.

16. The composition of claim 13, wherein the granules of dicalcium malate have a particle size ranging from about 50 microns to about 1500 microns.

17. The composition of claim 13, wherein the granules have a bulk density of at least 0.70 g/mL.

18. The composition of claim 13, wherein the composition comprises no more than 5.0 wt % silica.

19. A dosage formulation comprising granules of the calcium citrate malate composition of claim 1.

20. The dosage formulation of claim 19, wherein the dosage formulation is a tablet comprising compressed granules of calcium citrate malate.

21. A dosage formulation comprising granules of the dicalcium malate composition of claim 7.

22. The dosage formulation of claim 21, wherein the dosage formulation is a tablet comprising compressed granules of dicalcium malate.

23. A dosage formulation comprising granules of the dimagnesium malate composition of claim 13.

24. The dosage formulation of claim 23, wherein the dosage formulation is a tablet comprising compressed granules of dimagnesium malate.
